(19) United States
(12) Patent Application Publication
(10) Pub. No.: US 2026/0117130 A1
(43) Pub. Date: Apr. 30, 2026

(54) DIVIDING WALL COLUMN FOR EFFICIENT HYDROCARBON SEPARATION

(71) Applicants: Minaz Ramjanali Makhania, Gurgaon (IN); Rahul Radhakrishna Pillai, Manvel, TX (US); Rajeev Ranjan, Dwarka (IN); Shouvik Sarkar, Uttar Pradesh (IN); Michael J. Tallman, Houston, TX (US)

(72) Inventors: Minaz Ramjanali Makhania, Gurgaon (IN); Rahul Radhakrishna Pillai, Manvel, TX (US); Rajeev Ranjan, Dwarka (IN); Shouvik Sarkar, Uttar Pradesh (IN); Michael J. Tallman, Houston, TX (US)

(73) Assignee: KELLOGG BROWN & ROOT LLC, Houston, TX (US)

(21) Appl. No.: 19/366,794

(22) Filed: Oct. 23, 2025

Related U.S. Application Data
(60) Provisional application No. 63/711,322, filed on Oct. 24, 2024.

(51) Int. Cl. C10G 55/06; B01D 3/14; B01J 8/24; C07C 7/09; C10G 67/02

(52) U.S. Cl. CPC C10G 55/06; B01D 3/141; B01J 8/24; C07C 7/09; C10G 67/02; C10G 2400/02; C10G 2400/20; C10G 2400/22

(57) ABSTRACT

A hydrocarbon separation system comprising a dividing wall column is disclosed. A catalytic olefins converter is configured to receive a hydrocarbon feed and to produce an effluent. The dividing wall column includes a feed section, top section, bottom section, and side draw section. The feed section is configured to receive and to partially fractionate C3 and hydrocarbon stream. The top section configured to fractionate top product components to produce the first hydrocarbon fraction process stream containing C1, C2-C4 hydrocarbons. The side draw section configured to fractionate the second hydrocarbon fraction process stream containing C5 rich stream hydrocarbons and the bottom section configured to fractionate bottom product components to produce cracked natural gasoline (NG) product stream. Corresponding process and apparatus for separation of hydrocarbon feed are disclosed.

### CROSS REFERENCE TO RELATED APPLICATION

This application claims priority to U.S. Provisional Patent Application having Ser. No. 63/711,322 filed on Oct. 24, 2024 which is incorporated by reference herein.

### FIELD OF THE INVENTION

The present disclosure relates to separation of hydrocarbons. More particularly, it relates to a dividing wall column (DWC) for separation of hydrocarbons in a catalytic olefins converter unit.

### BACKGROUND OF THE INVENTION

Olefins are a class of chemicals such as ethylene, propylene, and butylene. The olefins are building blocks for a wide variety of products such as plastics, rubbers, and solvents. The olefins are produced from natural gas liquids and refinery products such as naphtha, kerosene, and gas oil. A wide variety of processes may be used to produce, recover, and convert the olefins. Olefins may be produced using various technologies such as, but not limited to, steam cracking, Fluid Catalytic Cracking (FCC) and so on. Further, the olefins may be recovered using light olefins recovery technology. Olefins are converted to higher valued products such as, but not limited to, polyethylene, polypropylene, and alkylate. Olefins may be converted to other olefins using Olefins conversion technology (OCT), ethylene dimerization, and comonomer production technology (CPT).

A conventional hydrocarbon separation process and the conversion of various feedstocks in a fluidized bed catalytic cracking reactor to olefins is illustrated in FIGS. 1A and 1B. An alkylation reaction/regeneration section 10 receives the naphtha, recycled C5, C4 and other feeds which are preheated and are fed to a FCC reactor 12 and cracking furnaces (not shown), where the larger hydrocarbons are broken catalytically (in the FCC reactor 12) and thermally (in furnaces) to produce preferentially olefin products along with other hydrocarbons.

The process gas from the FCC reactor 12 and furnaces is cooled in direct quench oil tower 16 and water quench tower 18. The process gas is then compressed using a process gas compressor 20, treated for oxygenates in an oxy wash tower 22, oxygen and acid gas is removal using caustic 24 and oxygen reactors 26. Further, the process gas is dried using dryer 28, 32 and sent forward for hydrocarbon separation as shown in FIG. 1A.

The process gas from a heat pumped (HP) Depropanizer 34 is treated to remove acetylene through acetylene remover 38 followed by cryogenic separation in chilling train 52 as shown in FIG. 1B. The hydrocarbon separation steps take place in the following order: heat pumped Depropanizer 34, Demethanizer 54, and Advance Deethanizer 60, low-pressure C2 Splitter 58, and a C3 Splitter 64. The C2 Splitter 58 is integrated with an ethylene refrigeration system 56.

The C4 and heavier bottoms from the Low Pressure Depropanizer 66 are fed to a C4 Rectifier 44 and Distillate Stripper 46 and to separate C4's and C5's which are recycled to the FCC reactor 10,12 to extinction. The C4 fraction can be exported as a liquid product if required. The bottom stream from the Distillate Stripper 46 is Aromatic Gasoline (C6+) that is used for Benzene extraction in other external units.

Gasoline-range hydrocarbons are partially condensed in the Water Quench Tower 18. A portion of this condensed gasoline is used as reflux in the upper section of the Oil Quench Tower 16 and Pyrolysis Fractionator and the balance is sent to the Distillate Stripper 46, along with condensed hydrocarbon from a Suction Drum (not shown) of the Process Gas Compressor 20 as shown in the FIG. 1A.

In the Distillate Stripper 46, the C3 and lighter hydrocarbons are stripped out and sent back to the Process Gas Compressor's 20 Suction Drum for recovery with the process gas. Reboiling duty for the Distillate Stripper 46 is provided by LP steam in the Distillate Stripper Reboilers.

A C5-rich vapor side draw is taken from the Distillate Stripper 46 and recycled back to FCC reactor 10,12. The Distillate Stripper 46 produces a C6+ gasoline rich bottoms stream that is rich in aromatics. The gasoline bottoms stream is pumped to external units.

A vapor side draw from the Distillate Stripper 46 and the bottom stream from the LP Depropanizer 66 are sent to the C4 Rectifier 44 which produces an overhead C4-rich vapor stream which is recycled back to the FCC reactor 12 or exported as crude C4 stream. The C4 Rectifier bottom stream is returned to the Distillate Stripper as shown in the FIG. 1A.

However, there are significant capital and maintenance costs with building a catalytic olefins unit in which the system for separation of hydrocarbons has this two column design i.e., distillate stripper 46/C4 rectifier 44, which can create an economic barrier to building systems for light olefins production.

It is always desirable to improve the hydrocarbon separation processes and systems by improving efficiency, reducing utility and energy requirements, enhancing plant safety, reducing capital requirements, reducing equipment footprint requirements, and/or improving the value of the products.

### SUMMARY

Examples of a Dividing Wall Column for efficient hydrocarbon separation as described herein can substantially obviates one or more of the problems due to limitations and disadvantages of the related art or at least to provide the public with a useful alternative.

In examples, the system and process described herein may provide improved separation or processing of hydrocarbon streams such as resulting from a catalytic cracking process designed or operated for obtaining increased amounts of light olefins.

In examples, the system and process as described may overcome one or more of the problems described above.

In examples, the functions of a distillate stripper and of a C4 rectifier of the prior art may be combined into a single fractionation column.

The prior art generally fails to provide the desired separation in an energy efficient manner and also requires higher capital to implement.

There is provided, in one non-limiting embodiment, a hydrocarbon separation system. The hydrocarbon separation system may include a catalytic olefins converter configured to receive a hydrocarbon feed and to produce an effluent. The hydrocarbon separation system may include a Distillate stripper and/or a C4 rectifier implementing a dividing wall column. The dividing wall column may include a feed section, a top section, a bottom section, and a side draw section, where the feed section may be configured to receive and to partially fractionate C3 and heavies hydrocarbon stream containing small amounts of C2 and lighter components (C2−). In this context, heavies hydrocarbon stream is considered as the one with more than 6 carbon atoms and light hydrocarbon stream is considered as the one with 1 to 6 carbon atoms. The top section may be configured to fractionate top product components from middle product components and to produce the first hydrocarbon fraction process stream containing C2 to C4 and lighter hydrocarbons. The side draw section may be configured to fractionate the second hydrocarbon fraction process stream containing C5 rich hydrocarbons and the bottom section may be configured to fractionate bottom product components from middle product components and to produce a cracked gasoline product stream.

In some embodiments, the C4 rectifier and/or the Distillate stripper implementing the dividing wall column may have an operating pressure from 3 bars to 4 bars.

In some embodiments, the dividing wall column may include a pre-fractionator section and a main fractionator section. In examples, the dividing wall column may include a bottoms product feed to a primary absorber.

In some embodiments, the dividing wall column may include an overhead C3/C4 product stream, a main-fractionator side stream that may be the aromatics-free C5/C6-rich product stream; and a bottoms product feed is directed to a hydrotreater.

In some embodiments, the bottoms product feed may be a cracked natural gasoline blendstock.

In examples, there is provided a process for separating hydrocarbon feed. The process may include a hydrocarbon feed to a catalytic olefins converter to produce an effluent and fractionating the effluent using a Distillate stripper and/or a C4 rectifier implementing a dividing wall column, wherein the dividing wall column having a feed section, a top section, a bottom section, and a side draw section. In examples, the process may include operating the Distillate stripper and/or a C4 rectifier with a dividing wall such that C3 and heavies hydrocarbon stream and also containing small amount of lighter components (C2 minus), passes from the feed section, a first hydrocarbon fraction process stream containing C2-C4 hydrocarbons passes from the top section, a second hydrocarbon fraction process stream containing C5 rich stream hydrocarbons passes from the side draw section, and a natural gasoline (NG) product stream passes from the bottom section.

In examples, there is provided an apparatus that may include a Distillate stripper and/or C4 rectifier implementing a dividing wall column. The dividing wall column may include a feed section, a top section, a bottom section, and a side draw section. The feed section may be configured to receive and to partially fractionate C3 and heavies hydrocarbon stream and also containing small amount of lighter components (C2 minus). The top section may be configured to fractionate top product components from middle product components and to produce the first hydrocarbon fraction process stream containing C2-C4 hydrocarbons. The side draw section configured to fractionate the second hydrocarbon fraction process stream containing C5 rich hydrocarbons and the bottom section may be configured to fractionate bottom product components from middle product components and to produce a natural gasoline (NG) product stream. Examples described herein may provide one or more of steady production of the light olefins, improvement on fractionation efficiency, reduced utility requirements, and reduced overall energy requirements.

Examples described herein may result in one or more of 20-30% less capital requirement as compared to the conventional column solution, less plot space (footprint) requirement. Thus, the embodiments herein may achieve the application of dividing wall column (DWC) for production of olefins that offers capital expenditure (CAPEX) benefits (less equipment count and plot space benefits) and operational expenditure (OPEX) benefits (lower energy requirement) as compared to the route shown in FIG. 1.

### BRIEF DESCRIPTION OF THE DRAWINGS

So that the recited features of the present invention can be understood in detail, a more particular description of the invention may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIGS. 1A and 1B are block diagrams illustrating a process of conventional separation scheme for catalytic cracking and recovery of hydrocarbons;

FIG. 2 is a block flow diagram illustrating separation of hydrocarbons by a dividing wall column, according to an embodiment as described herein;

FIG. 3 is schematic representation of a dividing wall separation column according to an embodiment as described herein;

FIG. 4 is an example schematic representation of a dividing wall separation column according to an embodiment as described herein; and

FIGS. 5A and 5B are block diagrams illustrating a dividing wall column being implemented in a separation scheme for catalytic cracking and recovery of hydrocarbons, according to an embodiment as described herein.

### DETAILED DESCRIPTION

A suitable heavy hydrocarbon feedstock can be catalytically cracked and the effluent resulting therefrom may be processed using a dividing wall column. In examples, it may be possible to produce or form one or more hydrocarbon product streams having desired purity of the hydrocarbon products. In examples, the product streams may be achieved in a manner that is more energy and capital cost efficient that existing processes.

It has been discovered that a dividing wall column can be used as a fractionator and simultaneously to separate various other products depending on FCC reactor effluent composition. Dividing wall column configurations which can be used as a fractionator in a catalytic olefins process are presented and discussed herein.

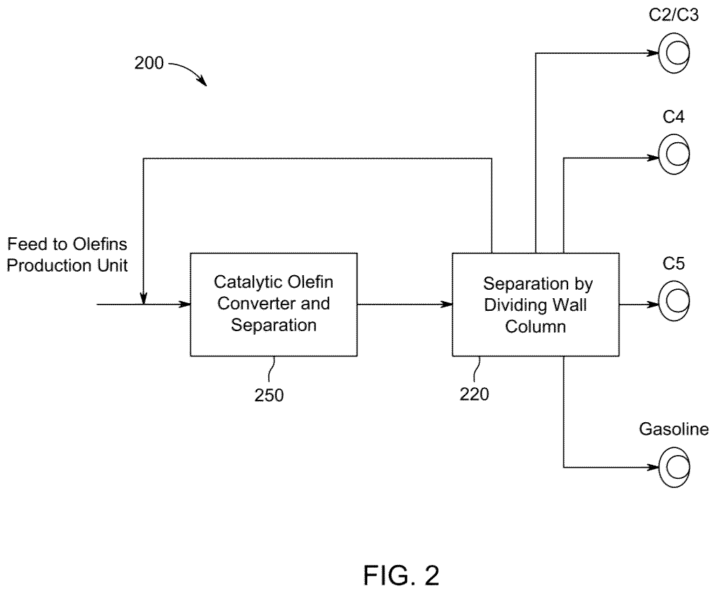

FIG. 2 is a block flow diagram illustrating separation of hydrocarbons by a dividing wall column as described herein. A hydrocarbon cracking system 200 as depicted in FIG. 2 may be configured for catalytic cracking of a heavy hydrocarbon feedstock and for obtaining selected hydrocarbon fractions from the effluent resulting therefrom, in accordance with one embodiment of the invention. It is to be understood that no unnecessary limitation to the scope of the claims which follow is intended by the following description. Those skilled in the art and guided by the teachings herein provided will recognize and appreciate that the illustrated system or process flow diagram has been simplified by the elimination of various usual or customary pieces of process equipment including some heat exchangers, process control systems, pumps, fractionation systems, and the like. It may also be discerned that the process flow depicted in the figure may be modified in many aspects without departing from the basic overall concept of the invention.

More specifically as shown in the FIG. 2 a hydrocarbon separation system 200 may include a catalytic olefin converter and separator 250 sometimes simply called a reactor herein, that receives olefin feed and produces an effluent ultimately to a dividing wall separation column 220 which separates the effluent. As mentioned, the olefin feed may be propylene, butylene, and/or amylenes (C5).

In some embodiments, the dividing wall column 220 may include a feed section, a top section, a bottom section and a side draw section.

A feed stream may be introduced into the column on the feed section of the dividing wall section of the column. The feed stream separates through repeated fractionation into at least three different product streams. One of these may be removed from the side draw section opposite the feed section. The other two product streams are removed near the top and bottom sections of the column similar to a conventional column configuration and operation.

The feed section may be one side of a bifurcated middle section. In examples, the feed section may be configured to be the side of a bifurcated middle section that receives the introduced feed. The upper portion of the feed section may be in direct fluid contact with the top section. In examples, the upper portion of the feed section may receive liquid from and/or pass vapor to the top section. The lower portion of the feed section may be in direct fluid contact with the bottom section. In examples, the lower portion of the feed section may receive vapor from and/or pass liquid to the bottom section.

In examples, the feed section may be configured to receive the feed stream and to make the initial separation of the top product components from the bottom product components using a feed distributor. In examples, the feed distributor may include one or more feed vapor chimneys for directing the vapor fraction from the introduced feed as well as allowing the vapor fraction passing up the feed section from lower in the column to move further up.

In an embodiment, the feed section of dividing wall column 220 may be configured to receive and to partially fractionate an introduced mixed C3 to C5+ hydrocarbon stream and also containing small amount of lighter components (C2−).

The top section may be configured to fractionate top product components from middle product components and to produce the first hydrocarbon fraction process stream containing C3 and lighters i.e., C1, C2, and C3 hydrocarbons and hydrogen.

The side draw section may be the other side that passes the middle product components of the dividing wall column. The upper portion of the side draw section may be in direct fluid contact with the top section. In examples, the upper portion of the side draw section may receive liquid from and passes vapor to the top section. The lower portion of the side draw section may be in fluid contact with the bottom section. In examples, the lower portion of the side draw section may receive vapor from and passes liquid to the bottom section. The side draw section may be configured to fractionate the second hydrocarbon fraction process stream containing C5 hydrocarbons.

The side draw section may be configured to receive the middle product components descending from the top section and ascending from the bottom section. As well, the side draw section may separate the middle product components from both the top product components that may drift down from the top section and the bottom product components that may rise up from the bottom section and the bottom section is configured to fractionate bottom product components from middle product components and to produce a cracked natural gasoline (NG) product stream.

In some embodiments, the C5 rich stream may be recycled back to reactor 250. Recycling of heavy C5-bearing streams, such as Light Coker Naphtha (LCN) may be performed for achieving incremental gains and valuable C3 and C4 olefins. Thus, the C5 rich streams are recycled back to the reactor 250. This recycling step ensures that more of the feed stream is fractionated and converted.

While FIG. 2 schematically illustrates the general process and system herein, there are more specific, non-limiting embodiments as will be discussed in the later parts of description.

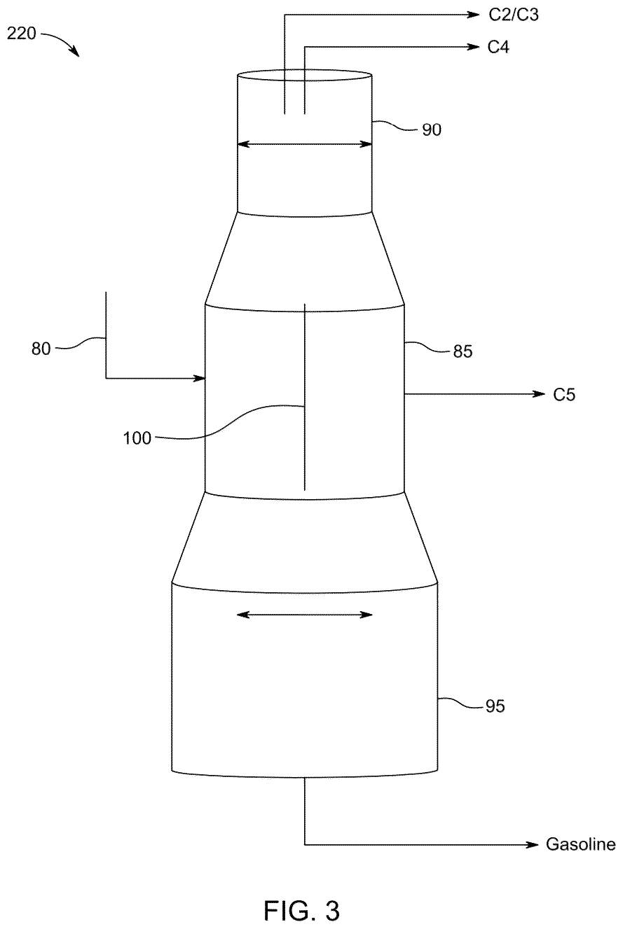

FIG. 3 is a simplified schematic representation of a dividing wall column 220 in accordance with one embodiment. In examples, the dividing wall column may be a distillate stripper and/or C4 rectifier. In examples, the dividing wall column 120 may include a dividing wall 100 positioned therewithin and may include a plurality of stages (not specifically shown). In examples, as shown in FIG. 3, the dividing wall column may include a central or middle dividing wall section 85 an upper or top section 90, and a lower or bottom section 95. In examples, central or middle dividing wall section 85 may include a bifurcated section. In examples, the central or middle dividing wall section 85 may include a feed section on one side of the bifurcated section and output stream section (i.e., for C5 stream) on the other side of the bifurcated section. As shown, the upper section 90 may have an internal width or diameter that is smaller than that of the central dividing wall section 85. As also shown, the lower section 95 may have an internal width or diameter that is greater than that of the central dividing wall section 85.

In examples, a mixed hydrocarbon feed stream can desirably be introduced via a line 80 into the central dividing wall section 85. A light fraction, C1, C2, C3 and mixed C4 in accordance with a preferred embodiment, can be withdrawn from the upper or top section 90. An intermediate fraction C5 in accordance with a preferred embodiment, can be withdrawn as a side product from the central or middle section 85, i.e., on the right side of the middle dividing wall section 85 as shown in the FIG. 3. A heavy fraction i.e., gasoline, in accordance with a preferred embodiment, can be withdrawn from the lower or bottom section 95.

Thus, through the incorporation and use of dividing wall column as herein described, the invention provides desired product splits for reactor effluent processing and does so in a generally more energy efficient manner than heretofore has been realized in the processing of such effluent streams.

It should be noted that other configurations and variations of the dividing wall column as shown in the FIG. 3 are possible. For example, the dividing wall column can be a Top dividing wall column and the dividing wall column can have different product draw offs and combinations thereof compared to the embodiment shown in the FIG. 3.

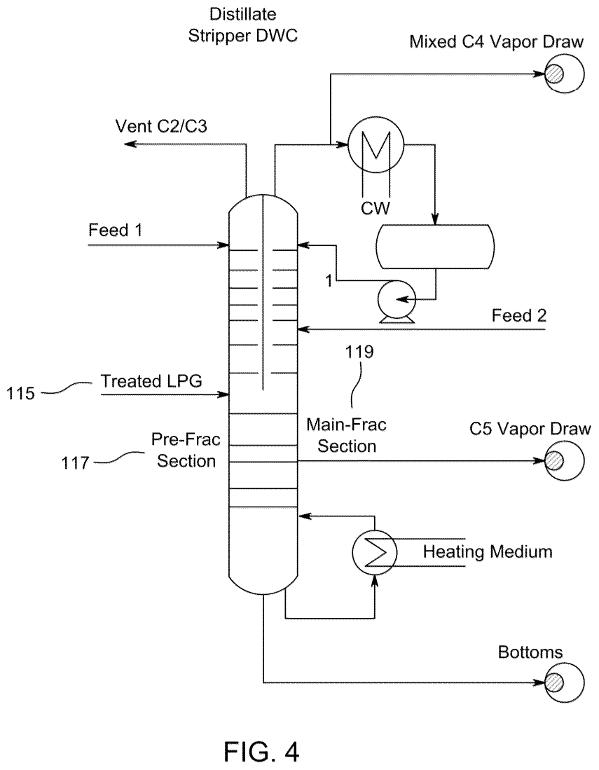

FIG. 4 is an example schematic representation of a dividing wall separation column according to an embodiment as described herein.

In some embodiments, the DWC 220 may include a pre-fractionator section 117 and a main fractionator section 119 divided by dividing wall. A reactor effluent containing a mixed C3 to C5+ components is fed to the DWC 220 as shown in the FIG. 4. The DWC 220 comprises a side draw that is the aromatics-free C5/C6-rich product stream and the DWC 120 comprises a side draw that is mixed C5 rich product stream and the DWC 220 also comprises a bottoms product which is heavy gasoline.

In some embodiments, the DWC 220 can be a middle dividing wall column that can be used to fractionate the hydrocarbons as described above.

In an example, the dividing wall column comprises an overhead C3/C4 product stream, a main-fractionator side stream that is the aromatics-free C5/C6-rich product stream and a bottoms product feed directed to a hydrotreater. The bottoms product feed is a cat-cracked naphtha gasoline blendstock.

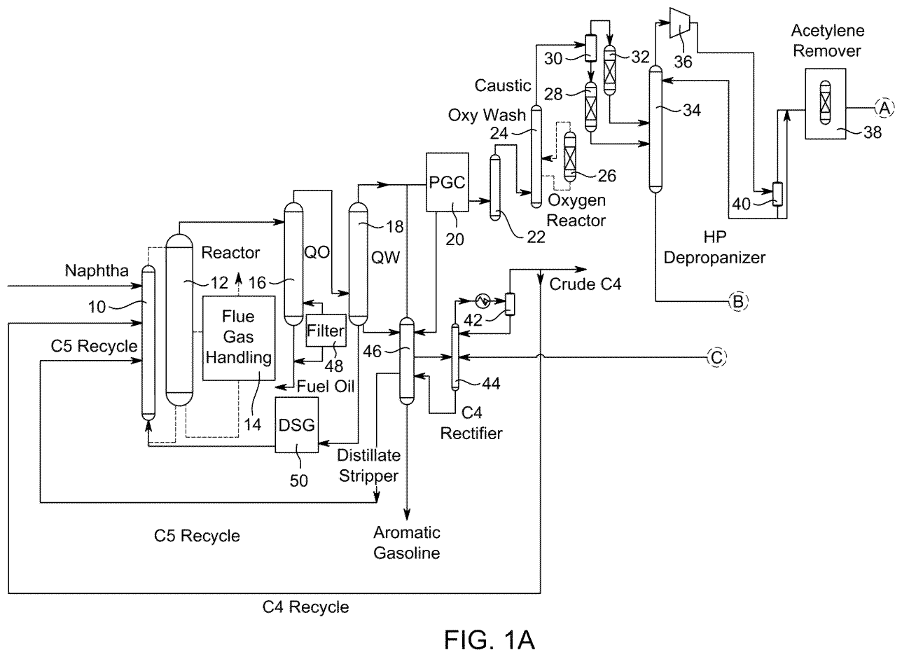

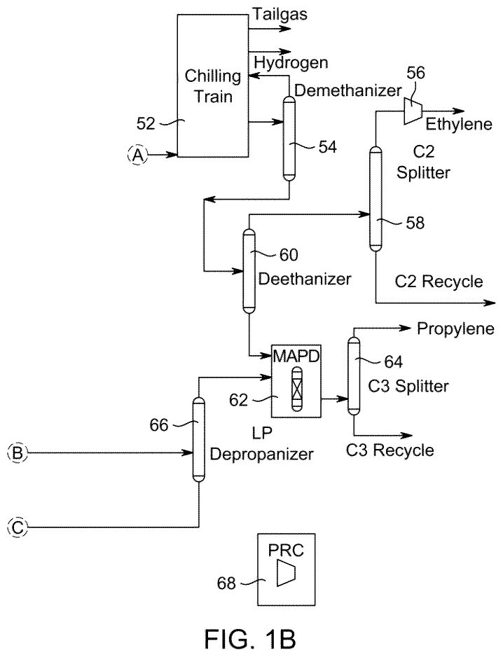

A simulation study was conducted to compare the performance of two conventional columns such as the distillate stripper column 46 and the C4 rectifier column 44 (as described in FIG. 1A) versus the dividing wall column. It has been observed that there is 21% reduction in total condenser duty and 16% reduction in total reboiler duty while using the divided wall column compared to the conventional two column design i.e., the distillate stripper 46 and the C4 rectifier 44 described in the FIG. 1A.

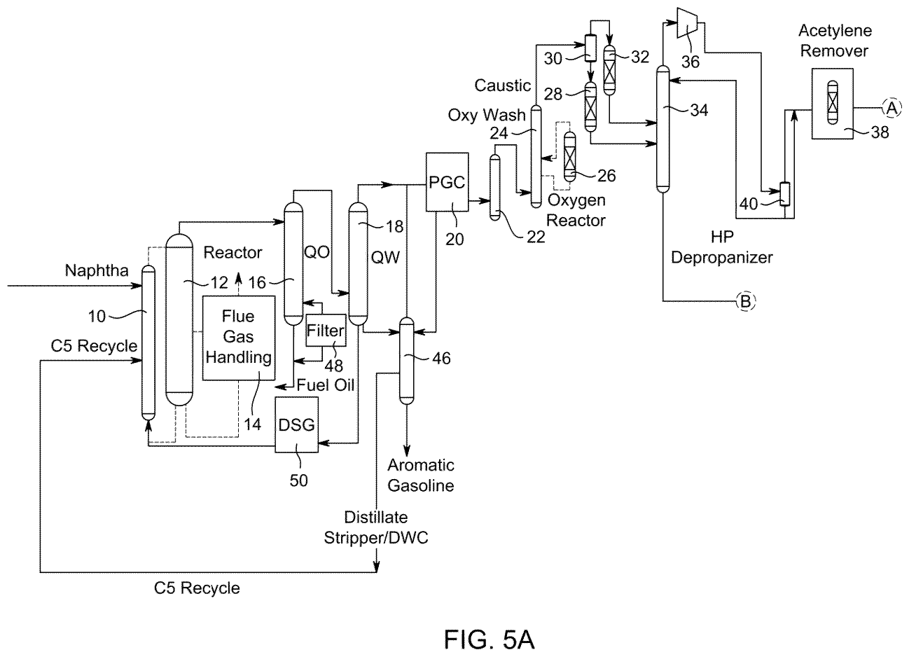

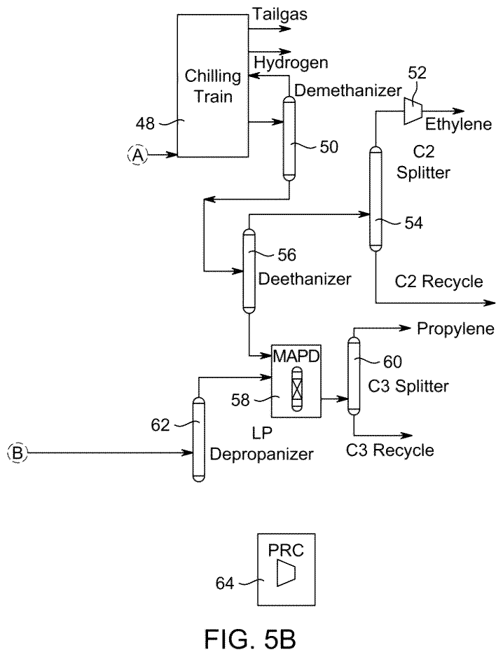

FIGS. 5A and 5B are block diagrams illustrating a dividing wall column being implemented in a separation scheme for catalytic cracking and recovery of hydrocarbons, according to an embodiment as described herein. The C4 rectifier 44 and the distillate stripper 46 as illustrated in the FIG. 1A are implemented as a single fractionation column 46 as shown in the FIG. 5A.

The FCC reactor can be used together with the dividing wall column to achieve one or of more of: improved efficiency, reduced utility and energy requirements, reducing CO₂ and/or NOx emissions, enhancing plant safety, reducing iso-butane loss, reducing capital requirements.

Thus, the dividing wall column along with FCC reactor performs the efficient hydrocarbon separation wherein the diving wall comprising the top section that may be configured to fractionate top product components from middle product components and to produce the first hydrocarbon fraction process stream containing C2 to C4 and lighter hydrocarbons. The side draw section may be configured to fractionate the second hydrocarbon fraction process stream containing C5 rich hydrocarbons and the bottom section may be configured to fractionate bottom product components from middle product components and to produce a cracked gasoline product stream. Therefore, the usage of the DWC together with FCC reactor as shown in the FIG. 5A achieves efficient separation of hydrocarbons. Unlike the existing systems which use two column design as shown in the FIG. 1A for hydrocarbon separation, the usage of the DWC together with FCC reactor can achieve significant capital gains and energy requirements, reduced CO₂ and/or NOx emissions, enhanced plant safety, reduced iso-butane loss, reducing capital requirements.

The single fractionation column is implemented as a dividing wall column as shown in the FIG. 5A. Thus, the dividing wall column is used together with FCC reactor for catalytic cracking of olefins and for efficient separation of hydrocarbons using the dividing wall column.

The Dividing Wall Column (DWC) technology can be used to further improve and simplify the process flow scheme (as shown schematically in FIGS. 5A and 5B) for olefins productions by the catalytic olefins converter unit i.e., the FCC reactor. The DWC delivers the performance of two conventional columns in series in a single column with reduced energy and capital requirement. Dividing Wall Column technology can be used to design a single column to replace the two columns i.e., the distillate stripper 46 and the C4 rectifier 44 as illustrated in FIG. 1A) in series system as shown in the FIG. 5A.

The single fractionation column 46 (which can be the distillate stripper 46 and/or the C4 rectifier 44) implements a dividing wall column having a feed section, a top section, a bottom section, and a side draw section. Thus, the distillate stripper 46 and/or the C4 rectifier 44 as illustrated in FIG. 1A are integrated to form a single fractionation column 46 which has a dividing wall. The top section is configured to fractionate top product components from middle product components and to produce the first hydrocarbon fraction process stream containing C1, C2, C3 and C4 hydrocarbons. The side draw section configured to fractionate the second hydrocarbon fraction process stream containing C5 hydrocarbons and the bottom section is configured to fractionate bottom product components from middle product components and to produce the cracked NG product stream.

In some embodiments, the C5 rich stream is recycled back to reactor 10 and 12.

In an example, the C4 rectifier 44 and/or the Distillate stripper 46 implementing the dividing wall column are operated under an operating pressure from 3 bars to 4 bars.

The words “comprising” and “comprises” as used throughout the claims, are to be interpreted to mean “including but not limited to” and “includes but not limited to”, respectively.

Certain embodiments and features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values are “about” or “approximately” the indicated value, and considers experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A hydrocarbon separation system comprising:
a catalytic olefins converter configured to receive a hydrocarbon feed and to produce an effluent;
a dividing wall column, wherein the dividing wall column having a feed section, a top section, a bottom section, and a side draw section,
where the feed section is configured to receive and to partially fractionate C3 and hydrocarbon stream and also containing small amount of lighter components (C2 minus),
wherein the top section is configured to fractionate top product components from middle product components and to produce a first hydrocarbon fraction process stream containing C2 to C4 hydrocarbons,
wherein the side draw section configured to fractionate a second hydrocarbon fraction process stream containing C5 hydrocarbons, and
where the bottom section is configured to fractionate bottom product components from middle product components and to produce a cracked natural gasoline (NG) product stream.

2. The hydrocarbon separation system of claim 1, wherein the dividing wall column having an operating pressure from 3 bars to 4 bars.

3. The hydrocarbon separation system of claim 1, wherein the dividing wall column comprising a pre-fractionator section and a main fractionator section; and the dividing wall column comprises a heavy gasoline bottoms product.

4. The hydrocarbon separation system of claim 3, wherein the dividing wall column comprises an overhead C3/C4 product stream, a main-fractionator side stream that is the aromatics-free C5/C6-rich product stream; and a bottoms product feed directed to a hydrotreater.

5. The hydrocarbon separation system of claim 4, wherein the bottoms product feed is a cracked natural gasoline blendstock.

6. A process for separating hydrocarbon feed, the process comprising:
introducing a hydrocarbon feed to a catalytic olefins converter to produce an effluent;
fractionating the effluent by implementing a dividing wall column, wherein the dividing wall column having a feed section, a top section, a bottom section, and a side draw section; and
operating the dividing wall column such that C3 and hydrocarbon stream passes from the feed section, a first hydrocarbon fraction process stream containing C2 to C4 hydrocarbons passes from the top section, a second hydrocarbon fraction process stream containing mixed C5 hydrocarbons passes from the side draw section, and a cracked natural gasoline (NG) product stream passes from the bottom section.

7. The process of claim 6, wherein the dividing wall column are operated with an operating pressure from 3 bars to 4 bars.

8. The process of claim 6, wherein the dividing wall column comprising a pre-fractionator section and a main fractionator section, and the dividing wall column comprises a heavy gasoline bottoms product.

9. The process of claim 8, wherein the dividing wall column comprises an overhead C3/C4 product stream, a main-fractionator side stream that is the aromatics-free C5/C6-rich product stream; and a bottoms product feed is directed to a hydrotreater.

10. The process of claim 9, wherein the bottoms product feed is a cracked natural gasoline blendstock.

11. An apparatus, comprising:
a dividing wall column, wherein the dividing wall column having a feed section, a top section, a bottom section, and a side draw section,
where the feed section is configured to receive and to partially fractionate C3 and hydrocarbon stream and also containing small amount of lighter components (C2 minus),
wherein the top section is configured to fractionate top product components from middle product components and to produce the first hydrocarbon fraction process stream containing C2 to C4 hydrocarbons,
wherein the side draw section configured to fractionate the second hydrocarbon fraction process stream containing mixed C4 hydrocarbons, and
where the bottom section is configured to fractionate bottom product components from middle product components and to produce a natural gasoline (NG) product stream.

12. The apparatus of claim 11, wherein the dividing wall column having an operating pressure from 3 bars to 4 bars.

13. The apparatus of claim 12, wherein the dividing wall column comprising a pre-fractionator section and a main fractionator section, and the dividing wall column comprises a bottoms product feed to a primary absorber.

14. The apparatus of claim 13, wherein the dividing wall column comprises an overhead C3/C4 product stream, a main-fractionator side stream that is the aromatics-free C5/C6-rich product stream; and a bottoms product feed directed to a hydrotreater.

15. The apparatus of claim 14, wherein the bottoms product feed is a cracked natural gasoline blendstock.
